(19) United States
(12) Patent Application Publication
(10) Pub. No.: US 2009/0043228 A1
(43) Pub. Date: Feb. 12, 2009

(54) LASER SHOCK PEENING OF MEDICAL DEVICES

(75) Inventors: CLAY W. NORTHROP, SALT LAKE CITY, UT (US); TED W. LAYMAN, PARK CITY, UT (US); TODD H. TURNLUND, PARK CITY, UT (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., MAPLE GROVE, MN (US)

(21) Appl. No.: 11/834,496

(22) Filed: Aug. 6, 2007

(51) Int. Cl. A61M 25/01

(52) U.S. Cl. 600/585

(57) ABSTRACT

A laser shock peening process for producing one or more compressive residual stress regions in a medical device is disclosed. A high-energy laser apparatus can be utilized to direct an intense laser beam through a confining medium and onto the target surface of a workpiece. An absorption overlay disposed on the target surface of the workpiece absorbs the laser beam, inducing a pressure shock wave that forms a compressive residual stress region deep within the workpiece. Medical devices such as stents, guidewires, catheters, and the like having one or more of these compressive residual stress regions are also disclosed.

### FIELD OF THE INVENTION

The present invention relates to medical devices and methods of manufacturing such devices. More specifically, the present invention pertains to laser shock peening of medical devices.

### BACKGROUND OF THE INVENTION

Medical devices such as stents, guidewires, catheters, intravascular filters, needles, and needle stylets are used in performing a wide variety of medical procedures within the body. To permit such devices to be inserted into relatively small regions such as the cardiovascular and/or peripheral anatomies, the various components forming the device must be made relatively small while still maintaining a particular performance characteristic within the body such as high flexibility and fatigue strength. In the design of stents, for example, it is desirable to make the struts highly flexible to permit the stent to be easily collapsed and inserted into a deployment device such as a sheath or catheter. The stent must also be resistant to the formation of cracks or other irregularities that can reduce the performance of the device. Crack propagation may occur, for example, in regions of the stent subjected to high tensile stresses such as at joints and bending regions. Repeated expansion and contraction of the device within the body may accelerate the growth of these cracks, reducing the performance of the device over time.

A number of processes have been used to impart flexibility and fatigue strength to the surface of medical devices. Such processes typically include treating the medical device by annealing, work hardening, or other suitable techniques to alter the physical characteristics of the material. In a shot peening process, for example, the surface of a workpiece is physically bombarded with particles or shot to form a superficial compressive residual stress region below the surface. The formation of these compressive residual stresses within the workpiece tend to negate the tensile stresses that can cause the initiation and growth of fatigue cracks, and allows the workpiece to undergo a greater amount of bending before plastically deforming.

While conventional processes such as shot peening have been used in treating medical devices, the efficacy of such processes are typically limited by the depth, and in some cases the accuracy, at which the compressive residual stress regions can be formed within the workpiece. In general, the greater the depth at which compressive residual stresses are formed within the workpiece, the greater the resistance to cracking that will result. Since many conventional processes such as shot peening are limited by the depth at which the compressive residual stress region can be formed, such processes are not always effective at preventing cracks in highly flexible regions deep within the surface of the workpiece.

### SUMMARY OF THE INVENTION

The present invention pertains to laser shock peening of medical devices. An illustrative laser shock peening process in accordance with an embodiment of the present invention includes the steps of providing a workpiece having a target surface to be irradiated, applying an absorption overlay onto the target surface, and directing a laser beam onto the absorption overlay to induce a pressure shock wave within the workpiece that can be used to produce one or more compressive residual stress regions therein. A high-energy laser apparatus capable of producing one or more intense laser beams may be provided to vaporize the absorption overlay material and form an interface layer of plasma above the target surface. The rapid expansion of volume and pressure at the interface layer induces a pressure shock wave within the workpiece that is greater than the dynamic yield stress of the workpiece material, creating a compressive residual stress region within the workpiece. In certain embodiments, a confining medium such as water can be provided to increase the magnitude of the induced pressure shock wave, further increasing the depth of the compressive residual stress region within the workpiece.

To form multiple compressive residual stress regions within the workpiece, a diffraction grating, prism or other similar device may be used to direct the light beam to selective locations on the workpiece target surface. In one illustrative embodiment, a holographic optical element may be employed to produce a desired laser beam pattern on the target surface of the workpiece. The holographic optical element may include a hologram that, when subjected to a laser beam, produces a desired pattern or array of compressive residual stress regions within the workpiece. In certain embodiments, for example, two adjacently pulsed laser beams can be directed simultaneously onto two locations of the target surface, inducing multiple pressure shock waves within the workpiece. The distance between the two locations on the target surface can be selected to produce a vertical compressive residual stress region deep within the workpiece formed by the overlapping of pressure shock waves. Other factors such as the laser beam intensity, duration, number of pulses, etc. may also be controlled to produce a desired compressive residual stress distribution within the workpiece.

In another illustrative laser shock peening process, multiple compressive residual stress regions may be formed within the workpiece by applying a patterned absorption overlay to the workpiece target surface. The patterned absorption overlay may comprise a patterned layer of absorptive paint, adhesive tape, or other suitable means for selectively absorbing the laser beam at certain locations above the target surface. When subjected to an intense laser beam, the patterned absorption overlay can be configured to induce multiple pressure shock waves that form a desired compressive residual stress distribution within the workpiece.

Using one or more of the aforesaid processes, a medical device such as a stent, guidewire, intravascular filter, guide catheter, needle, needle stylet, etc. may be formed having one or more compressive residual stress regions therein. In one illustrative embodiment, for example, a stent having a number of struts may include one or more compressive residual stress regions formed therein. In use, the compressive residual stress regions increase the flexibility and fatigue strength of the material at these locations, allowing the use of thinner struts with less disruption to the bloodstream. In another illustrative embodiment, a guidewire may include a core wire with one or more compressive residual stress regions formed in a pattern along the length of the guidewire, or within the entire guidewire. In certain embodiments, the one or more compressive residual stress regions may be formed about a joint used to fuse various components of the guidewire together. In use, the compressive residual stress regions can be used to impart one or more desired characteristics to the guidewire such as increased fatigue life and resistance to plastic deformation. In another illustrative embodiment, a medical device such as a guidewire, catheter, or the like, may include an elongated structure, such as a tube or wire, including a plurality of slots formed therein, wherein the elongated structure includes at least one compressive residual stress region.

### BRIEF DESCRIPTION OF THE DRAWINGS

FIG. 1 is a diagrammatic view showing an illustrative laser shock peening process for use in producing a compressive residual stress region within a workpiece;

FIG. 2 is a diagrammatic view showing the formation of a single compressive residual stress region within the workpiece of FIG. 1;

FIG. 3 is a diagrammatic view showing the formation of a vertical compressive residual stress region within a workpiece using another illustrative laser shock peening process;

FIG. 4 is a diagrammatic view of a holographic optical element configured to produce a pattern or array of compressive residual stress regions within a workpiece;

FIG. 5 is a diagrammatic view of another holographic optical element configured to produce a linear array of compressive residual stress regions within a workpiece;

FIG. 6 is a diagrammatic view showing a patterned absorption overlay that can be used to form multiple compressive residual stress regions within a workpiece;

FIG. 7 is a flat layout view of an illustrative tubular stent having a number of compressive residual stress regions formed therein;

FIG. 8 is an enlarged view of a portion of the stent shown in FIG. 7;

FIG. 9 is a flat layout view of another illustrative tubular stent having a number of compressive residual stress regions formed therein;

FIG. 10 is an enlarged perspective of a portion of the stent shown in FIG. 9;

FIG. 11 is a perspective view of an illustrative guidewire having a number of compressive residual stress regions formed therein;

FIG. 12 is an enlarged view of a portion of the guidewire shown in FIG. 11;

FIG. 13 is a perspective view of another illustrative guidewire having a compressive residual stress region formed about a joint;

FIG. 14 is an enlarged view showing the joint of FIG. 13;

FIG. 15 is a perspective view of another illustrative guidewire having a compress residual stress region formed about a joint;

FIG. 16 is an enlarged view showing the joint of FIG. 15;

FIG. 17 is a diagrammatic view showing the formation of a number of indents on a mandrel using an illustrative laser shock peening process;

FIG. 18 is a cross-sectional view along line 18-18 of FIG. 17, showing the circumferential arrangement of the indents about the mandrel;

FIG. 19 is another cross-sectional view showing the indented mandrel of FIG. 17 disposed within an extrusion die;

FIG. 20 is a cross-sectional view showing the profile of an illustrative tubular member extruded from the indented mandrel and die of FIG. 19;

FIG. 21 is a perspective view illustrating an embodiment of a medical device in accordance with the present invention for insertion in vasculature in anatomy;

FIG. 22 is an isometric view of a section of one embodiment of a tubular member in accordance with the present invention containing slots formed therein, wherein the segments between the slots include a compressive residual stress region;

FIG. 23 is an isometric view of a section of one embodiment of a tubular member in accordance with the present invention containing slots formed therein, wherein the tubular member includes regions having compressive residual stresses;

FIG. 24 is a partial cross-sectional side view of a guidewire including a slotted tubular member wherein the tubular member includes regions having compressive residual stresses; and

FIG. 25 is a partial cross-sectional side view of a catheter including a slotted tubular member wherein the tubular member includes regions having compressive residual stresses.

### DETAILED DESCRIPTION OF THE INVENTION

The following description should be read with reference to the drawings, in which like elements in different drawings are numbered in like fashion. The drawings, which are not necessarily to scale, depict selected embodiments and are not intended to limit the scope of the invention. Although examples of construction, dimensions, and materials are illustrated for the various elements, those skilled in the art will recognize that many of the examples provided have suitable alternatives that may be utilized.

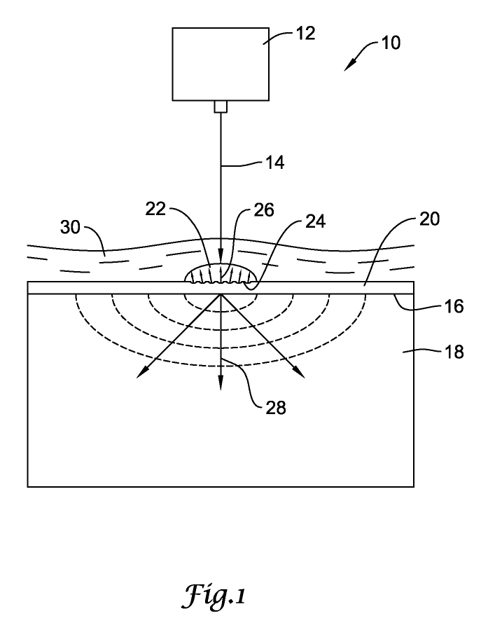

FIG. 1 is a diagrammatic view showing an illustrative laser shock peening process for use in producing a compressive residual stress region within a workpiece. The laser shock peening process, represented generally by reference number 10, includes a high-energy laser apparatus 12 configured to direct an intense laser beam 14 onto the target surface 16 of a metallic workpiece 18. The workpiece 18 may comprise one or more components of a stent, guidewire, catheter, intravascular filter, or other medical device wherein characteristics such as flexibility and fatigue strength are desirable. In certain embodiments, for example, the workpiece 18 may comprise a sheet or tube of material used in the construction of a stent, guidewire, catheter, or the like, or a core wire used in the construction of an intravascular guidewire, or the like.

The inventive techniques described herein can be used to form any number of devices having a metal, metal-polymer, or metal-metal composition, or materials including a carbon ceramic material and/or ceramic coatings. Examples of suitable metals include, but are not limited to, copper, aluminum, titanium, nickel, platinum, tantalum, nickel-titanium alloy, and steel-based alloys such as stainless steel. Composites of one or more of these materials may also be used, if desired.

A sacrificial absorption overlay 20 disposed over the target surface 16 of the workpiece 18 may be employed to absorb the laser beam 14 irradiated from the high-energy laser apparatus 12. The absorption overlay 20 may comprise one or more materials that are substantially opaque to laser radiation. The absorption overlay 20 may include, for example, a layer or sheet of paint (e.g. iron oxide or carbon), pentacrythritol tetranitrate (PETN), bismuth, aluminum, iron, lead, cadmium, tin, zinc, graphite, or other suitable material. In certain embodiments, a biocompatible absorption overlay 20 including carbon or high-density polytetrafluoroethylene (HDPTFE) loaded with tungsten filler may be employed. Adhesive or gel materials that are opaque to laser radiation may also be used in certain embodiments.

In addition to absorbing radiation from the laser beam 14, in some embodiments the absorption overlay 20 acts as a thermal barrier to protect the workpiece 18 from thermal effects generated during the laser peening process in some cases. The ability to prevent the transfer of heat into the workpiece 18 is important to maintain the desired performance characteristics of the material in some embodiments. With respect to some shape-memory nickel-titanium alloys, for example, the absorption overlay 20 prevents undesired thermal effects within the material that can alter the memory and/or flexibility characteristics of the material.

To induce a pressure shock wave within the workpiece 18, the high-energy laser apparatus 12 should be configured to provide an intense laser beam in some cases. In one illustrative embodiment, a high-energy laser apparatus may include a 600-Watt neodymium-doped glass laser capable of producing a 20-nanosecond laser beam pulse having an energy density of about 200 J/cm². The resultant shock wave produced by the high-energy laser apparatus 12 may have a pressure of greater than 1 GPa, which is above the yield stress of most metals.

When irradiated with the intense laser beam 14, the target surface 16 of the metallic workpiece 18 instantly vaporizes, forming an expanding gas release of plasma 22 at interface 24, which is then further heated by the incident laser beam 14. As the high-temperature plasma is formed at the interface 24, its pressure is increased to a range of about 1 to 10 GPa. This increase in temperature and pressure causes the plasma 22 to expand in a direction indicated generally by the upwardly pointed arrows 26, inducing a pressure shock wave within the workpiece 18. As indicated by the downwardly directed arrows 28, the induced pressure shock wave then propagates in part into the interior of the workpiece 18 along a semi-circular wavefront.

In certain embodiments, a confining medium 30 transparent to the irradiated laser beam 14 can be used to increase the magnitude of the induced pressure shock wave, in some cases by a factor of 5 or more in comparison to an open-air condition. The confining medium 30 may comprise any number of suitable materials known in the art, including, for example, water, glass, quartz, sodium silicate, fused silica, potassium chloride, sodium chloride, polyethylene, fluoropolymers, and nitrocellulose. The confining medium 30 may be formed integral with the absorption overlay 20, or may comprise a separate layer located adjacent to the absorption overlay 20.

As the induced pressure shock wave is transmitted into the workpiece 18, the region beneath the shocked area undergoes both plastic and elastic deformations, forming compressive residual stresses deep within the workpiece 18. The formation of compressive residual stresses within the workpiece 18 can be used to impart one or more desired characteristics to the medical device such as increased elasticity (i.e., resistance to plastic deformation) and resistance to cracking. Other characteristics such as corrosion resistance and wear resistance can also be achieved using a laser shock peening process.

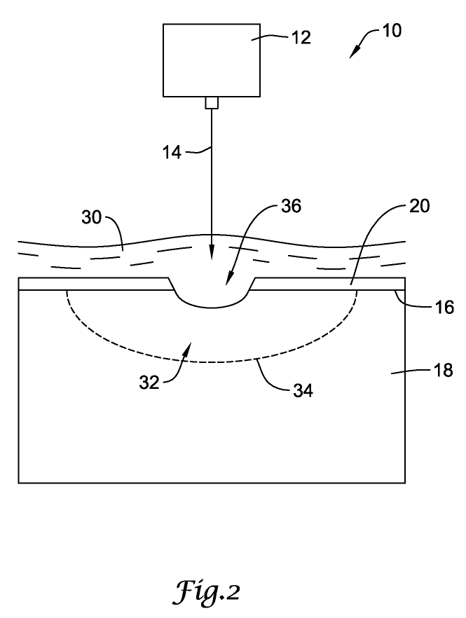

FIG. 2 is a diagrammatic view showing the formation of a single compressive residual stress region 32 within the workpiece of FIG. 1. As indicated by the semi-circular dashed line 34 in FIG. 2, the compressive residual stress region 32 may extend from an indent 36 formed on the target surface 16 of the workpiece 18 to a location deep within the interior of the workpiece 18. In certain embodiments, for example, the above process can be used to form a compressive residual stress region at a depth of about 0.05 to 0.1 inches or greater into the workpiece 18.

The magnitude and depth of the compressive residual stress region 32 can be controlled by the amount of energy delivered to the irradiated area, and the dwell time of the laser beam 14. In some cases, the amount of energy delivered to the irradiated area is governed by the power at which the beam is generated, by any attenuation of the laser beam, by the degree of beam focusing, and by the spatial characteristics of the laser beam. By increasing the intensity of the laser beam 14, for example, the magnitude of the induced pressure shock wave can be increased to provide greater compressive residual stresses within the workpiece 18. Other characteristics such as the acoustic impedance of the workpiece 18 material(s) may also have an effect on the magnitude and depth at which compressive residual stresses are formed in the workpiece 18.

The laser apparatus 12 can be configured to emit either a continuous or pulsed laser beam 14 onto the target surface 16 of the workpiece 18. In a pulsed laser beam configuration, the dwell time can be controlled by varying the pulse duration and frequency of the emitted beam. A similar result can be obtained with a continuous laser beam configuration through the use of a mechanical or optical shutter. All other factors being the same, an increase in dwell time results in the formation of compressive residual stress regions of greater magnitude and depth. Thus, by altering the pulse duration and/or frequency of the laser beam, a desired compressive residual stress distribution can be achieved within the workpiece 18.

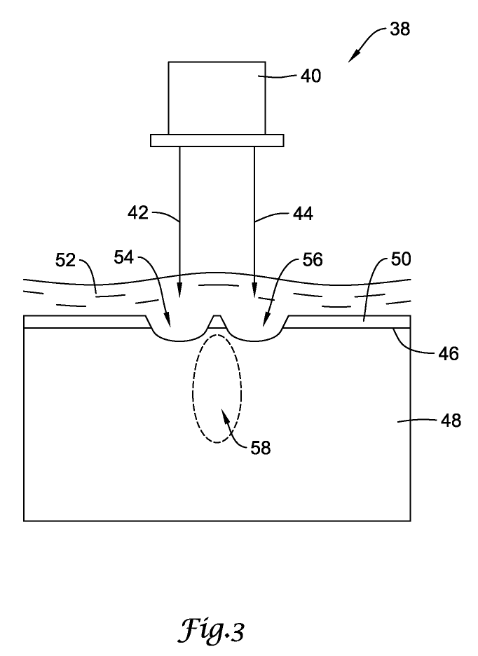

FIG. 3 is a diagrammatic view showing the formation of a vertical compressive residual stress region within a workpiece using another illustrative laser shock peening process. The laser shock peening process, represented generally by reference number 38, includes the use of a high-energy laser apparatus 40 that directs two intensive laser beams 42,44 onto the target surface 46 of a workpiece 48. As with other embodiments described herein, the workpiece 48 may comprise one or more components of a stent, guidewire, catheter, intravascular filter, or other medical device. A sacrificial absorption overlay 50 disposed over the target surface 46 of the workpiece 48 may be utilized to absorb the two irradiated laser beams 42,44. A confining medium 52 of water or other suitable transparent material may also be used to increase the magnitude of the induced pressure shock wave.

The high-energy laser apparatus 40 can be configured to simultaneously pulse the two laser beams 42,44 through the confining medium 52 and onto the absorption overlay 50. The intensity of each laser beam 42,44 can be made sufficient to induce two separate pressure shock waves within the workpiece 48, each emanating from a location immediately below the respective laser beam 42,44. As the pressure shock wave travels through the workpiece 48, first and second indents 54,56 are formed on the target surface 46 of the workpiece 48.

As is further indicated by dashed lines in FIG. 3, a vertical compressive residual stress region 58 located immediately below the midpoint of the first and second indents 54,56 can be formed within the workpiece 48. At this region 58, the two pressure shock waves induced by the two laser beams 42,44 overlap and collide to form a highly concentrated compressive residual stress region 58 within the workpiece 48. The shape and depth of the region 58 is dependent in part on the spacing between the two laser beams 42,44, and the magnitude of the induced pressure shock waves. As is discussed in greater detail below with respect to several illustrative medical devices, one or more of these vertical compressive residual stress regions 58 can be used to impart characteristics such as increased elasticity and fatigue strength to selective portions of the medical device, in some cases allowing smaller components to be used.

The laser beams 42,44 may be produced using multiple laser sources, or through the use of a single laser source in conjunction with a diffraction grating, prism, or other similar device. In certain embodiments, for example, the high-energy laser apparatus may include a type of diffraction grating called holographic optical element (HOE), which can be used to spatially modulate a single laser beam to produce a desired pattern onto the surface of the workpiece.

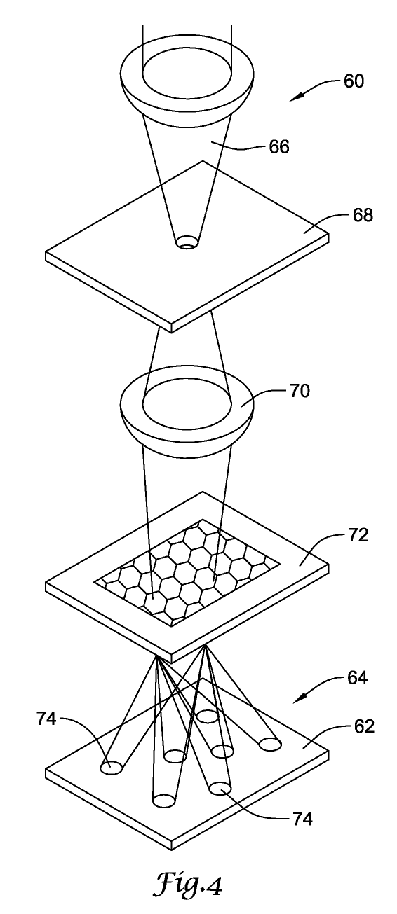

FIG. 4 is a diagrammatic view of an illustrative holographic optical element 60 that can be used to produce a desired laser beam pattern onto the target surface 62 of a workpiece 64. As shown in FIG. 4, the holographic optical element 60 may include a laser beam 66, a simple aperture mask 68, a transfer lens 70, and a hologram 72. As the laser beam 62 is received from the transfer lens 70, it is spatially modulated by the hologram 72, directing multiple frequency components of the laser beam onto the target surface 62 of the workpiece 64. The spatial distribution of these components can be adapted to provide a desired pattern or array on the target surface 62. In the illustrative embodiment of FIG. 4, for example, the holographic optical element 60 is configured to produce a complex pattern of indents 74 at various locations on the target surface 62. The indents 74 may include a pattern of dots, lines, or other desired geometrical shape. In use, these indents 74 form compressive residual stresses deep within the workpiece 64 that can be used to impart greater flexibility and fatigue strength to the medical device.

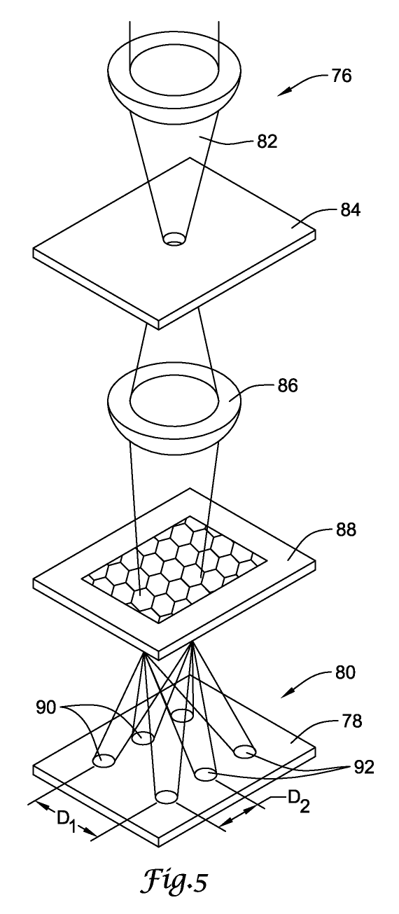

FIG. 5 is a diagrammatic view of another illustrative holographic optical element 76 configured to produce a longitudinal pattern or array of compressive residual stress regions onto the target surface 78 of a workpiece 80. As with the embodiment of FIG. 4, the holographic optical element system 76 may include a laser beam 82, a simple aperture mask 84, a transfer lens 86, and a hologram 88. In the illustrated embodiment of FIG. 5, however, the hologram 88 can be configured to produce two lines of indents 90,92 on the target surface 78. Each line may be spaced apart by a distance D₁ on the target surface 78, with each adjacent indent 90,92 on a particular line being spaced apart a distance D₂ with respect to each other.

In certain embodiments, the distance D₁ between each line of indents 90,92 can be selected to produce multiple vertical compressive residual stress regions within the workpiece 80. In the embodiment depicted in FIG. 5, for example, multiple vertical compressive residual stress regions may be formed within the workpiece 80 along a line substantially parallel and midway between the two lines of indents 90,92. The vertical compressive residual stress regions may be formed, for example, by spacing the indents 90,92 an appropriate distance D₁ apart sufficient to cause the induced pressure shock waves to overlap and collide. The distance D₂ between each adjacent indent 90,92 on a line may also be selected to cause overlap of the pressure shock waves, further increasing the amount of compressive residual stress imparted to the workpiece 80. Thus, by selecting distances D₁ and D₂ to produce multiple overlapping pressure shock waves, a desired compressive residual stress distribution can be formed within the workpiece 80.

The formation of multiple pressure shock waves within the workpiece can also be accomplished through the use of a patterned absorption overlay that is adapted to selectively absorb the laser pulse at only certain locations above the workpiece target surface. In certain embodiments, for example, a patterned absorption overlay of black paint can be applied to the workpiece. Using laser micro-texturing techniques known in the art, a pattern of absorptive dots, lines or other desired geometric pattern can be created on the absorption overlay. An inkjet patterning technique can also be employed in certain embodiments, if desired. When subjected to a large area laser beam, the patterned absorption overlay can be configured to produce multiple pressure shock waves within the workpiece at the absorptive regions of the overlay. As with other embodiments herein, the intensity, duration, and arrangement of the absorptive pattern can be selected to produce a desired compressive residual stress distribution within the workpiece.

In one such embodiment depicted in FIG. 6, a high-energy laser apparatus 94 employing a single laser source can be configured to produce multiple pressure shock waves within the target surface 96 of a workpiece 98 using a strip of patterned adhesive tape 100 for the absorption overlay. The patterned adhesive tape 100 may include an adhesive backing that allows the adhesive tape 100 to be applied directly to the surface 96 of the workpiece 98 with no gaps.

As further shown in FIG. 6, the patterned adhesive tape 100 may include a number of absorptive dots 102 configured to absorb a portion of the laser beam irradiated from the high-energy laser apparatus 94. The absorptive dots 102 can be spaced apart from each other by a transparent region 104 of the patterned adhesive tape 100, which unlike the absorptive dots 102, does not absorb the radiation emitted from the laser apparatus 94. In use, the high-energy laser apparatus 94 can be configured to emit a large area laser beam through a transparent confining medium (not shown) and onto the patterned adhesive tape 100. As the laser beam is irradiated onto the patterned adhesive tape 100, the absorptive dots 102 absorb the laser beam, inducing a number of pressure shock waves that can be used to form a desired compressive residual stress distribution within the workpiece 98.

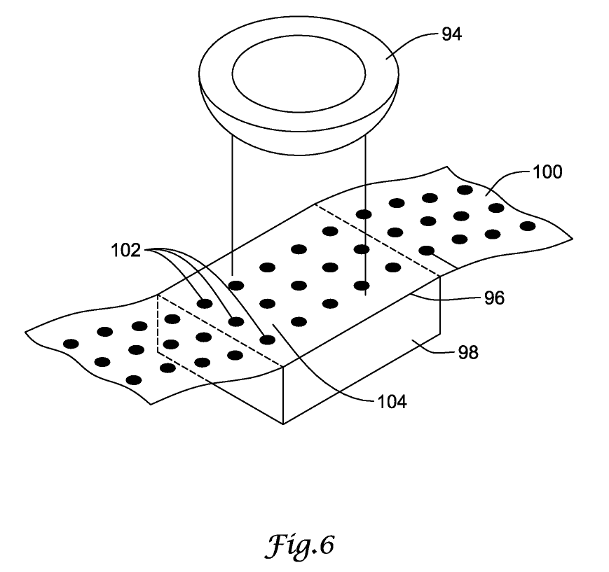

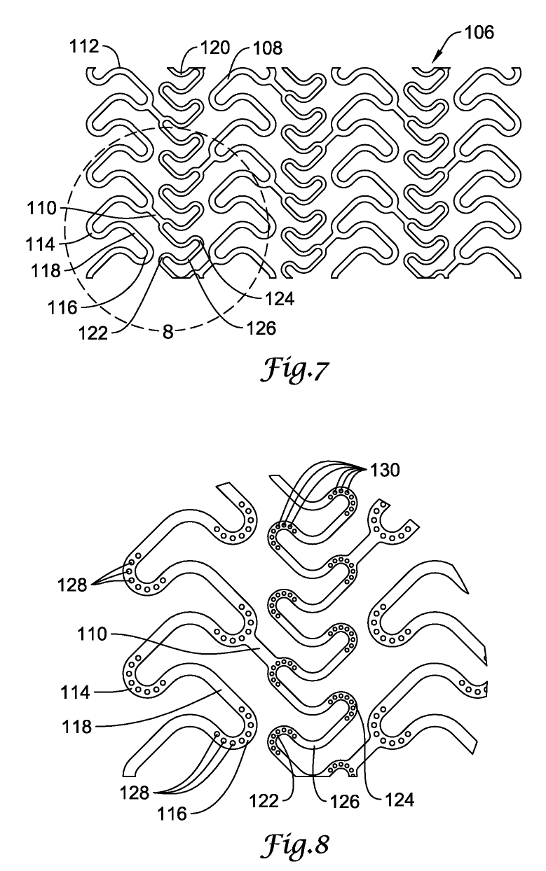

FIG. 7 is a flat layout view of an illustrative stent 106 having a number of compressive residual stress regions formed therein. The stent 106 may include a number of circumferential struts 108 that are connected to each other at various joints 110. The circumferential struts 108 may include first circumferential bands 112 having a first number of alternating first peaks 114 and first troughs 116 joined by bent struts 118. The first circumferential bands 112 may be joined at the joints 110 to second circumferential bands 120 having a second number of alternating second peaks 122 and second troughs 124 joined by bent struts 126. Together, the first and second circumferential bands 112,120 define a pathway around the periphery of the stent 106, forming a tubular structure that can be expanded within a body lumen.

To impart greater elasticity and fatigue strength, a number of compressive residual stress regions may be formed at selective locations of the stent 106 normally subjected to relatively high tensile stresses. As shown in greater detail in FIG. 8, for example, a number of indents 128 may be created by laser shock peening one or more selective peaks 114,122 and/or troughs 116,124 of the first and second circumferential bands 112,120, forming multiple compressive residual stress regions within the thickness of the stent 106 at these locations. In similar fashion, a number of indents 130 may be formed on one or more of the joints 110, forming multiple compressive residual stress regions within the thickness of the stent 106 at the joints 110. In use, these compressive residual stress regions can be used to prevent the growth or acceleration of cracks, nicks, pits, or other irregularities that can reduce the fatigue life of the stent 106. Moreover, the compressive residual stress regions can be used to increase the elasticity of the stent 106, in some cases allowing the use of thinner struts with less disruption to the bloodstream. In certain embodiments, the formation of compressive residual stress regions on the stent 106 can be used to provide texture to the stent surfaces as a final step after, for example, electropolishing, thereby reducing the contact area and friction of the stent 106 within the delivery device.

As can be further seen in FIG. 8, each of the indents 128,130 may be closely spaced apart from each other along the length of each band 112,120. With respect to the indents 128 formed on the peaks 114,122 and/or troughs 116,124 of each band 112,120, for example, the indents 128 can be spaced apart from each other along a line located centrally on the thickness of the bands 112,120, forming compressive residual stress regions deep within the surface of the bands 112,120. The indents 128,130 can be arranged in any pattern or array to produce a desired compressive residual stress distribution within the stent 106. In certain embodiments, for example, a laser shock peening process utilizing two or more simultaneous laser beams may be utilized to form multiple vertical compressive residual stress regions within the stent 106. As with other embodiments described herein, the depth and magnitude of the vertical compressive regions may be controlled by varying the number, intensity, and duration of the laser beam pulses.

When a biocompatible absorption overlay is utilized (e.g. carbon or HDPTFE), the process of laser shock peening the stent 106 can be accomplished after the stent 106 has been crimped on the delivery system (e.g. a balloon catheter). The remaining portion of the absorption overlay not used during the laser shock peening process can then be implanted within the body while still being attached to the stent 106. By selectively peening one or more regions of the stent 106 in this manner, the inherent stresses caused by the compression of the stent 106 on the delivery device can be either reset, or altered in some other desired manner. In certain embodiments, higher securement forces can also be imparted to the crimped stent 106 by laser shock peening the stent 106 after it has been placed on the delivery device.

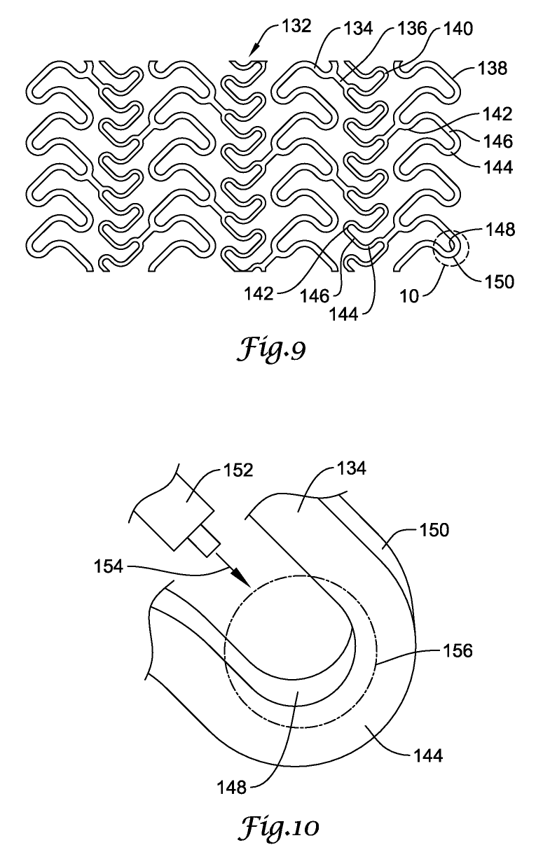

FIG. 9 is a flat layout view of another illustrative stent 132 having a number of compressive residual stress regions formed therein. Stent 132 may be configured similar to stent 106 described above, including a number of circumferential struts 134 that are connected to each other at various joints 136. The circumferential struts 134 may include a number of alternating first circumferential bands 138 and second circumferential bands 140, each including a number of alternating peaks 142 and troughs 144 joined by bent struts 146. The peaks 142 and troughs 144 may each include a U-shaped bend or other similar shape. In use, the shape of the peaks 142 and troughs 144 facilitates expansion of the stent 132 from a relatively small profile when disposed on a delivery device (e.g. a stent delivery catheter) to a larger profile during implantation within the body. In certain embodiments, for example, the struts 134 can be configured to radially expand via a balloon catheter that can be inflated to expand the stent 132 within a blood vessel. In an alternative embodiment, the stent 132 can be configured to self-expand when placed within a blood vessel, if desired.

During expansion of the stent 132 within the body, the amount of stress within the first and second circumferential bands 138,140 may increase significantly. In those embodiments in which the stent 132 is configured to expand using a balloon catheter, for example, the interior portion 148 of each peak 142 and trough 144 may undergo a significant increase in tensile stress in comparison to the outer portion 150 resulting from the decrease in the radius of curvature at this region. As a result, small cracks or other irregularities can form, reducing the performance characteristics desired in the device. Repeated expansion and contraction of the device caused by the pumping action of the heart can accelerate the growth of the cracks, reducing the performance of the stent 132 over time.

To impart greater elasticity and fatigue strength at these regions, the interior portion 148 of the peaks 142 and/or troughs 144 can be laser shock peened to form one or more compressive residual stress regions therein. As can be seen in FIG. 10, for example, a high-energy laser apparatus 152 similar to laser apparatus 12 discussed above can be configured to direct an intense laser beam 154 onto the interior portion 148, inducing a shock wave within the width of the strut 134 that forms a compressive residual stress therein. The area at which the laser beam 154 is focused onto the strut 136 can be altered to either increase or decrease the size of the treatment area, as desired. In the illustrative embodiment depicted in FIG. 10, for example, the laser beam 154 is configured to treat a relatively large area of the strut 134 all at once, as is indicated generally by the region delineated by the dash lines 156. It should be understood, however, that the amount of laser focusing as well as other characteristics of the laser apparatus 152 could be controlled to produce other desired flexibility characteristics within the stent 132. Moreover, while FIG. 10 illustrates the treatment of only one of the troughs 144, it should be understood that other peaks 142 and/or troughs 144 could be similarly treated as discussed herein. In one illustrative method, for example, the laser apparatus 152 can be configured to treat one section of the stent 132, and then index to another region of the stent 132 (e.g. an adjacent peak 142 or trough 144) to treat a subsequent section, and so forth.

While the embodiments of FIGS. 7-10 illustrate the treatment of selective locations of the stent, the present invention is not limited as such. In certain embodiments, for example, it may be desirable to laser shock peen the whole stent to induce compressive residual stresses within the entire structure. In one illustrative method, a high-energy laser apparatus having a large area laser beam can be utilized to treat the entire stent structure at once. A focusing/defocusing lens or other such device can be employed to adjust the area of the incident laser beam to accommodate stents of varying size and construction, if desired.

FIG. 11 is a perspective view of a guidewire 158 having a number of compressive residual stress regions formed therein. Guidewire 158 may include a tapered core wire 160 having a spiraled band 162 of compressive residual stress regions formed therein by a laser shock peening process. The spiraled band 162 may wrap around the outer periphery of the tapered core wire 160 along all or a portion of its length.

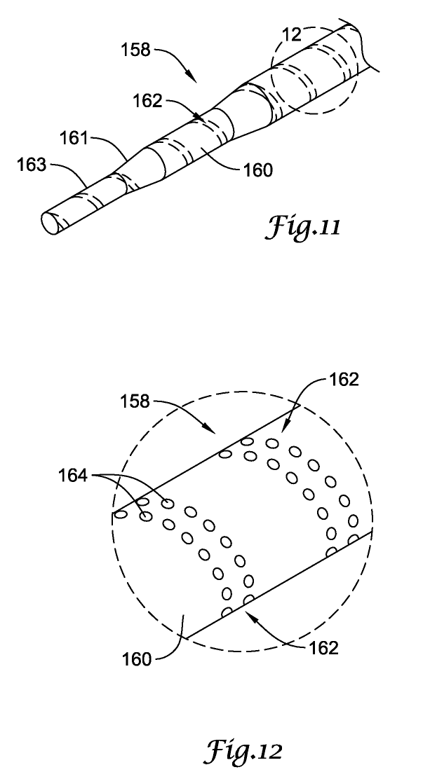

As shown in greater detail in FIG. 12, the spiraled band 162 may include a number of indents 164 formed at an angle with respect to the longitudinal axis of the guidewire 158. The indents 164 can be formed, for example, by simultaneously emitting two adjacent laser beams onto the surface of the core wire 160, and then rotating and advancing the core wire 160 relative to the two laser beams. In an alternative embodiment, the core wire 160 can be held stationary and the laser apparatus rotated and advanced along the length of the core wire 160 to produce the desired pattern. A combination of these techniques may also be used to produce the desired spiral band 162 structure of FIG. 11. In one such embodiment, for example, the core wire 160 can be rotated while the high-energy laser apparatus is advanced along the length of the core wire 160.

In use, the indents 164 create a compressed plane of residual stresses at an angle to the guidewire 158 that can be used to impart greater elasticity and torqueability to the guidewire 158. While two adjacently disposed lines of indents 164 are specifically illustrated in FIG. 12, it should be understood that other alternative methods could be utilized to form compressive residual stresses within the guidewire 158. In one alternative embodiment, for example, two simultaneous laser beams can be configured to strike the surface of the core wire 160 at opposite sides (i.e. 180° alpha) apart from each other. The two laser beams can be configured to produce two separate pressure shock waves within the guidewire that collide to form a compressive residual region within the middle of the guidewire 158. In another alternative embodiment, the laser apparatus can be configured to peen the whole guidewire 158, forming compressive residual stresses within the entire structure, if desired.

When manufacturing guidewires like guidewire 158, core wire 160 may be formed from a generally metallic shaft (e.g., stainless steel, such as 304V, 304L, and 316

LV stainless steel; nickel-titanium alloy including linear elastic and/or super elastic nitinol; etc.) that is ground, for example, using a known centerless or other suitable grinding technique to define one or more tapers and/or a tapered section 161 as depicted in FIG. 11. In general, tapered section 161 is disposed adjacent a distal section 163 of core wire 160 and may reduce the cross-sectional size of core wire 160 to that of distal section 163. For example, core wire 160 may taper to a size of about 0.001 to about 0.010 inches in diameter or cross-sectional width at distal section 163. In some instances, if the distal portion 163 of the core wire 160 that is to be ground to a small diameter is not at least partially annealed before grinding, the wire will not remain straight after grinding. This is due to residual stresses within the wire that are imparted by previous process steps such as drawing and straightening. However, the distal section 163 of the core wire 160 may have a lower yield strength after annealing, which reduces the core wire's fatigue strength and resistance to plastic deformation while in use. One approach to addressing this issue may be to cold work core wire 160, after annealing and grinding, in a manner that allows distal section 163 to remain straight and resistant to plastic deformation while in use. Because of the relatively-small size, it may be difficult to cold work distal section 163 of core wire 160 so that it is and/or remains adequately straight. One possible means for cold working a small diameter wire such as distal section 163 of core wire 160 is through the use of a laser peening process similar to those described herein. Alternatively, it may be possible to grind the core wire 160 without annealing beforehand, then fixture the distal section 163 so that it is straight (e.g., by putting it in tension) and use a laser peening process to impart residual compressive stresses to “set” the distal section 163 in the straight condition.

Accordingly, it may be desirable to utilize laser shock peening along portions or all of core wire 160 in a manner similar to what is described herein when manufacturing guidewires like guidewire 158. For example, laser shock peening may be utilized along portions or all of distal section 163. This may create one or more compressive residual stress regions in distal section 163 that are similar to, for example, compressive residual stress region 162. The compressive residual stress regions in distal section 163 may improve the ability of distal section 163 to remain straight after grinding and to be resistant to plastic deformations during the use of guidewire 158. It should be noted that laser shock peening of core wire 160 is not intended to be limited to being in any particular pattern such as the spiral pattern depicted in FIGS. 11-12 as any suitable pattern may be utilized including laser shock peening along any portion of all of distal section 163.

FIG. 13 is a perspective view of another illustrative guidewire 166 having a compressive residual stress region formed about a joint. Guidewire 166 may include a proximal section 168, a tapered section 170 located distally of the proximal section 168, and a distal section 172 located further distally of the tapered section 170. Guidewire 166 may have a composite structure formed by one or more different materials that can be selected to improve characteristics such as torquability, pushability and flexibility. In one illustrative embodiment, for example, the proximal section 168 of the guidewire 166 may comprise a material different than that of the tapered section 170 and distal section 172, forming a composite guidewire that changes in flexibility along its length. In certain embodiments, for example, the proximal section 168 may comprise a relatively stiff material such as stainless steel, whereas the tapered and distal sections 170,172 may comprise a relatively flexible material such as Nitinol.

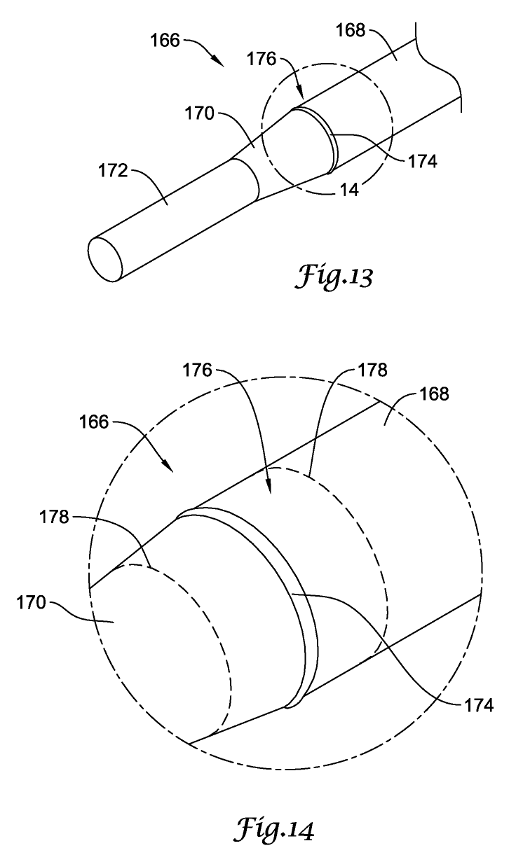

As can be further seen in FIG. 13, a weld joint 174 or other similar bonding means may be utilized about the outer periphery of the guidewire 166 to fuse the proximal section 168 to the tapered section 170. Depending on the particular welding technique employed, cracks or other irregularities may be introduced at the location of the weld joint 174, reducing the performance characteristics of the device. To prevent crack propagation, a compressive residual stress region 176 may be formed about the joint 174 by laser shock peening the outer periphery of the guidewire 166. As indicated by dashed lines 178 in FIG. 14, the compressive residual stress region 176 may comprise a circumferential band that extends about the guidewire 166 at the region of the joint 174. In use, the formation of the compressive residual stress region 176 at this region increases the flexibility and strength of the joint 174.

FIG. 15 is a perspective view of another illustrative guidewire 180 having a compressive residual stress region formed about a joint. Guidewire 180 is similar in construction to guidewire 166, having a proximal section 182, a tapered section 184 located distally of the proximal section 182, and a distal section 186 located further distally of the tapered section 184. In the illustrative embodiment of FIG. 15, guidewire 180 further includes a spring coil 188 and atraumatic distal tip 190, which can be used to facilitate insertion of the guidewire 180 through the tortuous anatomy.

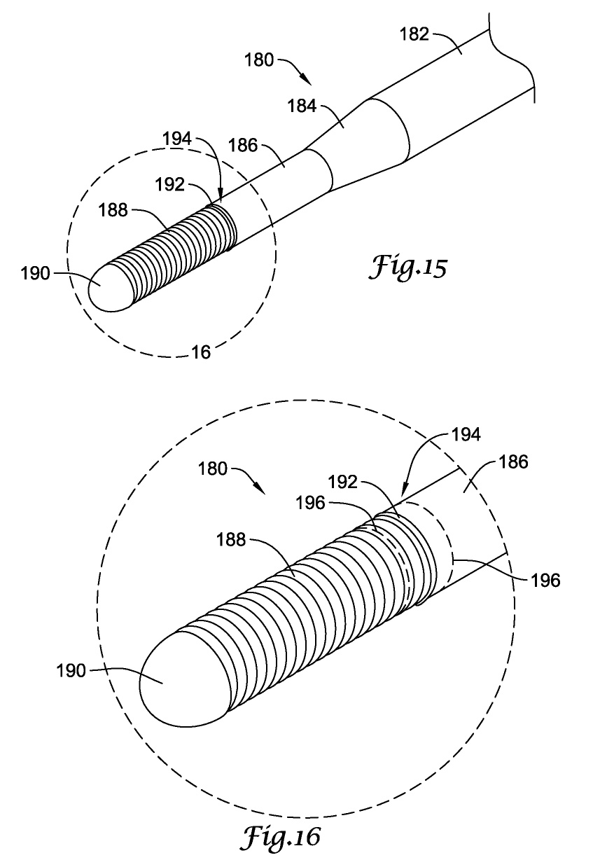

Attachment of the spring coil 188 to the distal section 186 of the guidewire 180 can be accomplished using a weld joint 192 or other suitable bonding means. To further strengthen the joint 192 and permit greater flexion of the guidewire 180, a compressive residual stress region 194 may be formed at or near the weld joint 192. As indicated by dashed lines 196 in FIG. 16, the compressive residual stress region 194 may comprise a circumferential band that extends about the guidewire 180 at the region of the joint 192.

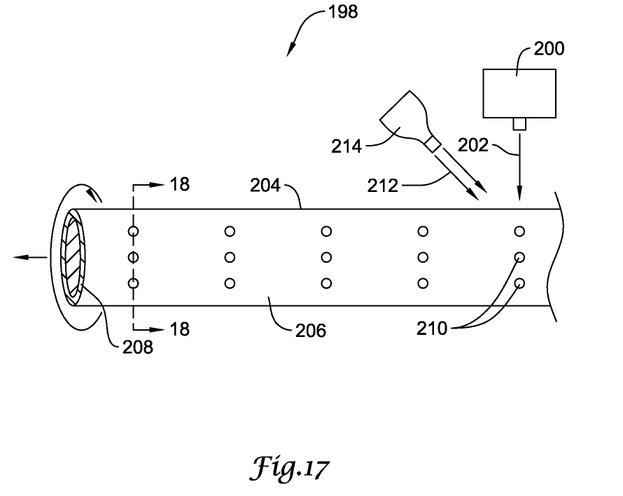

Turning now to FIGS. 17-20, a laser shock peening process for producing a tubular member having a number of internal ridges will now be described. The process, represented generally by reference number 198 in FIG. 17, may begin with the step of providing a high-energy laser apparatus 200 configured to direct an intense laser beam 202 onto the target surface 204 of a metallic mandrel 206. In the illustrative embodiment depicted in FIG. 17, the metallic mandrel 206 has a circular profile which, when used in an extrusion die, can be used to form a tubular member having a circular interior. It is contemplated, however, that the interior may have any number of desired shapes.

A sacrificial absorption overlay 208 may be applied to the target surface 204 of the mandrel 206. The absorption overlay 208 may include one or more materials that are substantially opaque to laser radiation, causing the absorption overlay 208 to absorb the laser beam 202 and form a number of indents 210 on the target surface 204. A confining medium may also be used to increase the magnitude of the induced pressure shock wave. In the illustrative embodiment of FIG. 17, for example, a jet of water 212 emitted from a nozzle 214 may be directed onto the target surface 204 of the mandrel 206 to form an acoustic barrier for the induced pressure shock wave.

With the laser apparatus 200 directed towards the mandrel 206, one or more laser beam 202 pulses can be directed onto the absorptive overlay 208 while rotating and periodically moving the mandrel 206 across the path of the laser beam 202. In an alternative configuration, the mandrel 206 can remain stationary while the high-energy laser apparatus 200 is rotated and periodically advanced across the surface of the mandrel 206. Using either embodiment, the indents 210 can be arranged in any pattern or array on the mandrel 206, as desired. In the illustrative embodiment depicted in FIG. 17, for example, the indents 210 are shown arranged in several circumferential bands along the length of the mandrel 206.

FIG. 18 is a cross-sectional view showing the indented mandrel 206 across line 18-18 of FIG. 17. As can be seen in FIG. 18, the indents 210 are formed circumferentially about the target surface 204 of the mandrel 206. For sake of clarity, only 8 indents 210 are shown about the mandrel 206. In actual practice, however, a greater or smaller number of indents 210 can be formed about the target surface 204, as desired.

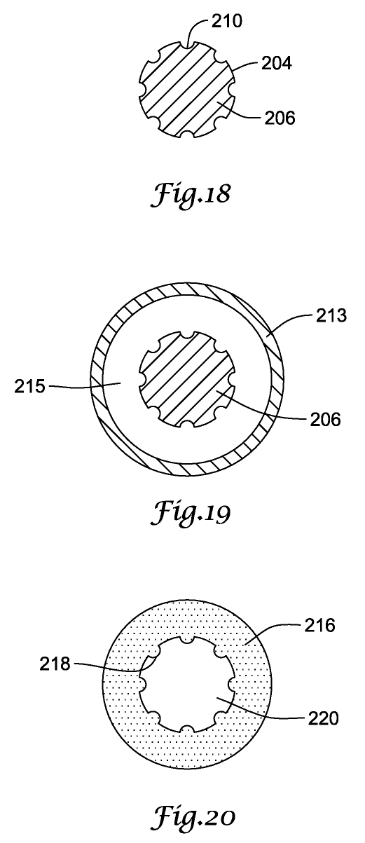

Once the desired pattern of indents 210 has been formed on the target surface 204, a tubular member is then created by extruding a polymeric material through a die using the indented mandrel 206. As can be seen in cross-section in FIG. 19, for example, the indented mandrel 206 can be placed within a circular extrusion die 213 to form a tubular member. The annular space 215 between the extrusion die 213 and indented mandrel 206 can be injected with a polymeric material that can be used to produce a tubular member having a number of internal ridges. As can be seen in FIG. 20, for example, the extrusion die 212 and indented mandrel 206 can be used to form a tubular member 216 having a number of internal ridges 218 disposed within its interior 220 corresponding in size and shape with the indents 210 formed on the mandrel 206. In use, these internal ridges 218 reduce the amount of friction within the interior 220 of the tubular member 216 as it is advanced over a guiding member such as a guidewire or guide catheter.

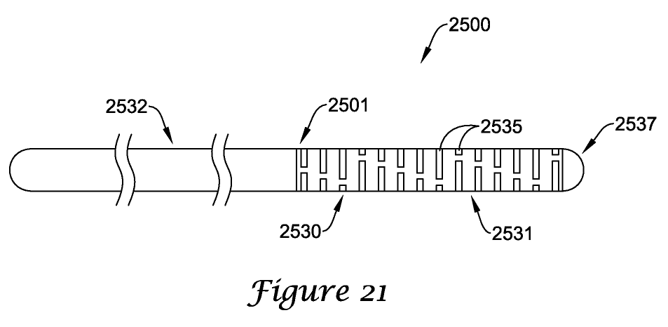

FIG. 21 illustrates another example of a medical device 2500 which may include structure having regions of compressive residual stresses, for example, formed by laser shock peening as discussed above. The medical device 2500 may be any of a wide variety of devices, but in this case is shown as a guidewire, or the like, including an elongate shaft 2501 including an elongated member 2530 having a plurality of grooves, cuts and/or slots 2535 that are formed in at least a portion thereof. For example, in the embodiment shown, the slots 2535 may be formed in a distal portion 2531 of the shaft 2501, while a proximal portion 2532 is substantially free of such slots. However, this is not intended to be limiting as any portion or the entire length of the shaft 2501 and/or member 2530 may include slots 2535. The slots 2535 may be formed and/or adapted to provide increased flexibility to a portion of the medical device 2500, such as the distal portion 2531, while still allowing for suitable torque transmission. The member 2530 may have a generally solid cross-section, such as a wire or ribbon, or the like, or may be a generally tubular member including a lumen extending therethrough.

The member 2530 may comprise or be made of a metallic material, for example, the metallic materials discussed above with regard to the other embodiments. For example, the member 2530, or other portions of the device 2500, may be comprise stainless steel, such as 304V, 304L, and 316LV stainless steel; nickel-titanium alloy including linear elastic and/or super elastic nitinol; or any other suitable metallic material. The medical device 2500 may include a distal tip 2537 disposed at the distal end of member 2530. As can be appreciated, the medical device 2500 may include additional structures, such as additional shaft sections, core wires and/or members, shaping structures, such as a shaping ribbon or wire, one or more coils, marker members, or the like, or other structures that may be used in constructing the device 2500, some of which will be shown and discussed in additional embodiments below. All or portions of the shaft 2501 and/or member 2530 may include regions of compressive residual stresses, for example, formed by laser shock peening as discussed above.

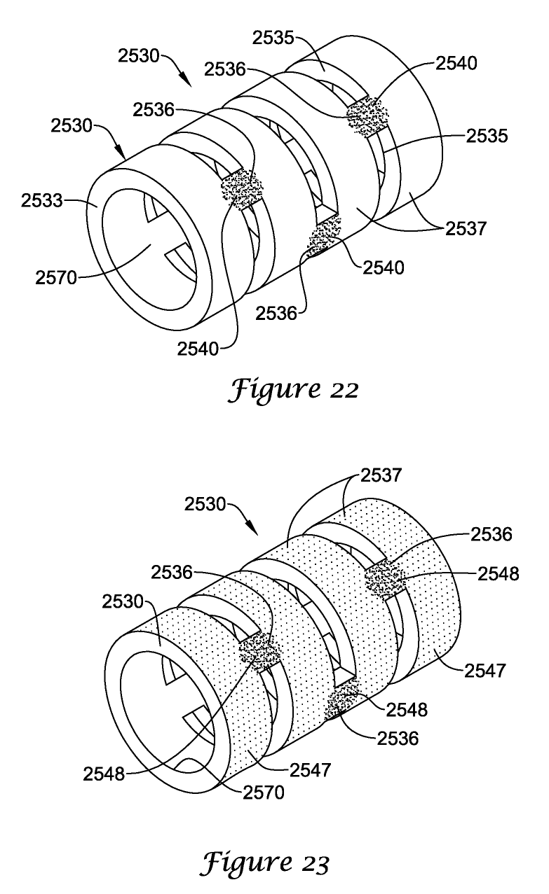

With reference now to FIG. 22, in some embodiments, the member 2530 may be a generally elongated tubular member 2530 having a lumen 2570 extending there through, and a plurality of slots 2535 formed therein. In some embodiments, at least some if not all of the slots 2535 may extend all the way through the wall 2533 of the member 2530, such that there is fluid communication between the lumen 2570 and the exterior of the member 2530 through the slots 2535. In other embodiments, however, some or all of the slots 2535 may extend only partially into the wall 2533, such that the slots 2535 may be more channel-like structures in the outer surface of the member 2530. The shape and size of the slots 2535 can vary, for example, to achieve the desired characteristics. For example, the shape of slots 2535 can vary to include essentially any appropriate shape, such as rectangular, pill-shaped, oval, or the like, and may include rounded or squared edges. Additionally, the size of the slots 2535 can be configured to provide the desired characteristics.

In some embodiments, at least some, if not all of the slots 2535 are disposed at the same or a similar angle with respect to the longitudinal axis of the member 2530. As shown, the slots 2535 can be disposed at an angle that is perpendicular, or substantially perpendicular, or on a plane that is substantially normal to the longitudinal axis of the member 2530. However, in other embodiments, one or more slots 2535 or groups of slots may be disposed at different angles relative to one or more other slots 2535 or groups of slots and/or relative to the longitudinal axis.

The slots 2535 may be formed such that the remaining structure of the member 2530 includes a plurality of turns and/or ring structures 2537 interconnected by one or more segments or beams 2536. In other words, such rings 2537 and beams 2536 may include portions of the member 2530 that remain after the slots 2535 are formed in the body of the member 2530. As shown in FIG. 22, two or more slots 2535 forming a group may be formed part way through tubular member 2530 at a point along the length of the member 2530, leaving an axial beam 2536 between the slots 2535 in the group and/or interconnecting two adjacent rings 2537. Such an interconnected ring structure may act to maintain the integrity of the tubular member 2530 and/or maintain a relatively high degree of tortional stiffness, while maintaining a desired level of lateral flexibility.

The slots 2535 and/or the associated rings 2537 and beams 2536 may be disposed in a pattern that provides the desired properties. For example, the slots 2535, or groups thereof, can be arranged along the length of, or about the circumference of, the member 2530 to achieve desired properties. For example, the slots 2535 can be arranged in a symmetrical pattern, such as being disposed essentially equally on opposite sides about the circumference of the member 2530, or equally spaced along the length of the member 2530, or can be arranged in an increasing or decreasing density pattern, or can be arranged in a non-symmetric or irregular pattern. As can be appreciated, the slots 2535 can be arranged in groups of two or more slots that are disposed at substantially the same point along the length of the member 2530. In some embodiments, some adjacent slots 2535 or groups of slots can be formed such that they include portions that overlap with each other about the circumference of the member 2530. In other embodiments, some adjacent slots 2535 or groups or slots can be disposed such that they do not necessarily overlap with each other. Other characteristics, such as slot size, slot shape and/or slot angle with respect to the longitudinal axis of the member 2530, can also be varied along the length of the member 2530, for example, to vary the flexibility or other properties. In other embodiments, moreover, it is contemplated that portions of the member 2530, or the entire member 2530, is substantially free of and/or does not include any such slots 2535.

Any of the above mentioned slots 2535 can be formed in essentially any known way. For example, slots 2535 can be formed by methods such as micro-machining, saw-cutting, laser cutting, grinding, milling, casting, molding, chemically etching or treating, or other known methods, and the like. In some such embodiments, the structure of the member 2530 is formed by cutting and/or removing portions of the member to form slots 2535. Some example embodiments of appropriate micromachining methods and other methods for forming slots, and structures for tubular members and medical devices including tubular members are disclosed in U.S. patent appliation Ser. No. 10/213,123 (now US Pub. No. 2003/0069522); and Ser. No. 10/604,504 (now US Pub. No. 2004/0181174-A2); and in U.S. Pat. Nos. 6,766,720; and 6,579,246, the entire disclosures of all of which are herein incorporated by reference. Some example embodiments of etching processes are described in U.S. Pat. No. 5,106,455, the entire disclosure of which is herein incorporated by reference.

As indicated above, all or portions of the shaft 2501 and/or member 2530, such as the remaining rings 2537 and beams 2536, or other portions of the member 2530 may include regions of compressive residual stresses, for example, formed by laser shock peening. As can be appreciated, in some embodiments, the beams 2536 may be somewhat small regions that are configured to transfer applied forces along the length of the tubular member 2530. Therefore, it may be beneficial to augment segments 2535 with increased elasticity and fatigue strength. As such, in some embodiments, one or more, or all of the beams 2536 may preferentially include compressive residual stress regions 2540. Compressive residual stress regions 2540 may be selectively formed at beams 2536, or compressive residual stress regions 2540 may be formed along other portions, or along substantially the entire length the portion including slots, such as distal portion 2531. Alternatively or additionally, compressive residual stress regions may be formed along any portion or substantially the entire length of the member 2530, the shaft 2501 and/or the medical device 2500. Compressive residual stress regions 2540 may be formed by a laser shock peening process such as those disclosed above.

Alternatively, and/or additionally, the member 2530 or other portions of the device 2500 can include two or more different regions of compressive residual stress that are at a different magnitude from one another. For example, as shown in FIG. 23, the member 2530 can include a first region of compressive residual stresses 2547 having a first magnitude of compressive residual stress that may include portions or substantially the entire length of member 2530 having slots 2535. A second region of compressive residual stresses 2548 having a second magnitude of compressive residual stresses may be located at and include one or more, or all of the beams 2536. For example, the first region of compressive residual stresses 2547 may be located at and include the rings 2537 and/or other portions of the member 2530, and the second region of compressive residual stresses 2548 may be located at and include one or more or all of the beams 2536. The second region of compressive residual stresses 2548 may have a magnitude of compressive residual stresses different from that of the first region of compressive residual stresses 2547. For example, the second region of compressive residual stresses 2548 may be greater than the magnitude of compressive residual stresses in the first region 2547. Therefore, beams 2536 may have increased elasticity and fatigue strength relative to adjacent portions of the tubular member 2530, such as the rings 2537. Therefore, slots 2535 may be cut to enhance flexibility of the medical device without compromising the integrity of the tubular member, and the use of regions of compressive residual stresses may enhance the characteristics of the member. Additionally and/or alternatively, due to the use of regions of compressive residual stresses within the beams 2536, the beams 2536 may be formed to include less material after cutting slots 2535, while still providing sufficient structural integrity to the tubular member. In some embodiments, as the amount of material forming the beams 2536 is reduced, the lateral flexibility characteristics may be increased.

As indicated above, compressive residual stress regions 2547, 2548 may be located at any portion of the member 2530. Compressive residual stress regions 2547, 2548 may be formed in a portion of the tubular member 2530 before the slots 2535 are formed, or after the slots 2535 have been formed along the tubular member 2530. In some embodiments, the compressive residual stress regions 2547, 2548 may be formed after the slots 2535 have been formed in the tubular member 2530 such that the process of forming slots 2535, such as micromachining, may not adversely affect the compressive residual stresses 2547, 2548 formed in the tubular member 2530. In some such cases, any stresses remaining as a result of the slot forming process may be reduced and/or removed during the subsequent laser shock peening process, wherein compressing residual stress regions 2547, 2548 are formed in the tubular member 2530.

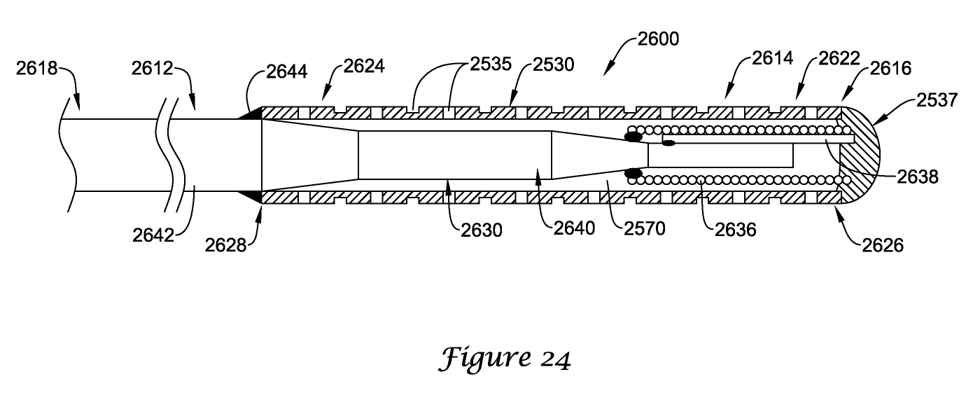

As can be appreciated, such a member 2530 may be incorporated and/or used in any of a wide variety of medical devices. For example, refer now to FIG. 24 which shows a partial cross-sectional view of a medical device 2600, such as a guidewire, that may include a slotted tubular member, such as the tubular member 2530, which includes one or more regions of compressive residual stresses. The guidewire 2600 can include a proximal region 2612, a distal region 2614, a distal end 2616, and a proximal end 2618. As used herein, the proximal region 2612 and the distal region 2614 may generically refer to any two adjacent guidewire sections along any portion of the guidewire 2600. The guidewire 2600 includes a generally tubular member 2530, for example, as discussed above. The tubular member 2530 includes a distal section 2622, a proximal section 2624, a distal end 2626, and a proximal end 2628. Again, the tubular member 2530 includes an inner lumen 2570, and may include a plurality of slots 2535 formed therein, and may include rings 2537 and beams 2536 for example, as shown in FIGS. 22 and 23, and may include one or more regions of compressive residual stresses, as discussed above.

A distal tip member 2537 may be disposed at the distal end 2626 of the tubular member 2530 and/or the distal end 2616 of the guidewire 2600. The distal tip member 2537 may be any of a broad variety of suitable structures, for example, a solder tip, a weld tip, a pre-made or pre-formed metallic or polymer structure, or the like, that is attached or joined to the distal end of the tubular member 2535 using a suitable attachment technique.

The guidewire 2600 may also include a core member 2630 that may be attached to the tubular member 2535, and extend from a location within the tubular member 2535 and/or from the proximal end 2628 of the tubular member 2535, for example, to the proximal end 2618 of the guidewire 2600. As can be appreciated, a portion of the core member 2630 may extend into at least a portion of the lumen 2570. In the embodiment shown, the core member 2630 includes a distal portion 2640 that extends within the lumen 2570, and a proximal portion 2642 that extends proximally from the tubular member 2530. In the embodiments shown, the core member 2630 ends proximally from the distal tip member 2537 and/or proximally of the distal end 2626 of the tubular member 2530. In other embodiments, however, core member 2630 may extend to, and be attached to the distal tip member 2537. The core member 2630 can be attached to the tubular member 2530 in any suitable manner and at any suitable location. For example, the core member 2630 may be attached to the tubular member 2530 through one or more attachment areas 2644, which in this embodiment are disposed adjacent the proximal end 2628 of the tubular member 2530. It can also be appreciated that the core member 2630 may be attached to the tubular member 2530 through the distal tip member 2537. It should be understood that additional attachment areas, and/or alternative positioning of attachment areas may be used in other embodiments.

Additionally, in other embodiments, the core member 2630 may be absent, and/or the tubular member 2530 may extend to the proximal end 2618 of the guidewire 2600. For example, in some other embodiments, the tubular member 2530 may extend along substantially the entire length of the guidewire 2600, for example, from the proximal end 2618 to the distal end 2616, and the core member 2630 may be present and disposed within at least a portion of the tubular member 2530, or may be absent, as desired.

The guidewire 2600 may also include other structures, such as such as a shaping wire or ribbon, one or more coils, marker members, coating, sleeve, or the like, or others, but such structures are not necessary in some other embodiments. In the embodiment shown, the guidewire 2600 includes a distal coil member 2636 and a shaping ribbon member 2638 that may be, for example, attached to and extend distally from the distal end of the core wire 2630, and may be attached, for example, to the tip member 2537. The materials used for such structures can be any that are suitable for their intended purpose, such as metals, polymers, or composites, and may include the example materials discussed above, or others. Additionally, the attachment of the various components can be achieved using any suitable attachment techniques, some examples of which may include adhesive bonding, welding, soldering, brazing, mechanical bonding and/or fitting, or the like, or any other suitable technique. As can be appreciated, this is but one example of a guidewire construction, and many others including various additional components and/or arrangements are contemplated.

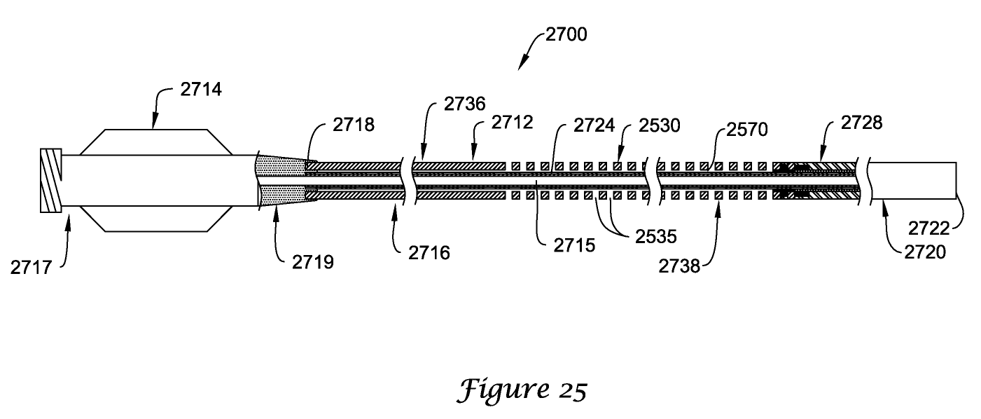

Refer now to FIG. 25 which shows a partial cross-sectional view of another medical device, in this case a catheter 2700, that may include a slotted tubular member, such as the tubular member 2530, or other structure which includes one or more regions of compressive residual stresses. The catheter 2700 can include an elongate shaft 2712 including a proximal portion 2716 having a proximal end 2718, and distal portion 2720 having a distal end 2722. As can be appreciated, the shaft 2712, or a portion thereof, can include a tubular member, for example, a slotted tubular member 2530, as discussed above. Again, the tubular member 2530 includes an inner lumen 2570, and may include a plurality of slots 2535 formed therein, and may include rings 2537 and beams 2536 for example, as shown in FIGS. 22 and 23, and may include one or more regions of compressive residual stresses, as discussed above. In the embodiment shown, the tubular member 2530 includes a distal portion 2738 including slots 2535 formed therein, and a proximal portion 2736 that is substantially free of such slots.

The shaft 2712 can also include an inner tubular member 2724 defining an inner lumen 2715. For example, the slotted tubular member 2535 may be used a reinforcing member for the shaft 2712, and the inner tubular member 2724 may extend within the slotted tubular member 2535. The catheter may also include a distal tip structure 2728 disposed about a distal portion of the inner tubular member 2724 and/or the slotted tubular member 2535. A manifold 2714 can be connected to the proximal end of the elongate shaft 2712, and include a lumen and/or other structure to facilitate connection to other medical devices (e.g., syringe, Y-adapter, etc.) and to provide access to the lumen within the shaft 2712. The manifold may include a hub portion 2717 and a strain relief portion 2719. In some embodiments, the shaft 2712 may include additional devices or structures such as inflation or anchoring members, sensors, optical elements, ablation devices or the like, depending upon the desired function and characteristics of the catheter 2700. The catheter 2700 may also include other structures, such as one or more coil or braid, marker member, coating, sleeve, or the like, or others, but such structures are not necessary in some other embodiments. As can be appreciated, this is but one example of a catheter construction, and many others including various additional components and/or arrangements are contemplated. Some example embodiments of catheter constructions incorporating a slotted tubular member are disclosed in U.S. patent application Ser. No. 10/400,750 (Publication No. US-2004-0193140-A1), which is incorporated herein by reference.

The various laser shock peening processes also may be used to alter the elasticity and/or the elastic behavior of any of the devices and/or device components described herein. For example, a laser shock peening process may be used to impart cold work to portions of all of a tubular member (including a slotted tubular member), a core wire, a stent, any of the various components of a guidewire, any of the various components of a catheter, any of the various components of other medical devices, combinations thereof, and the like, including any of those structures described herein; thereby altering the elastic behavior. The relative amount of cold work imparted by laser shock peening processes that results in changes in elastic behavior may be the same as those sufficient to define compressive residual stress regions or it may be different. For example, a greater degree of laser shock peening may be necessary to alter the elastic behavior of structure than that required to define compressive residual stress regions. In some embodiments, cold working in the range of 5 to 70%, or in the range of 10 to 60% may be imparted using laser shock peening. However, other amounts of cold work outside of these ranges is contemplated, depending upon the desired characteristics. By altering the elastic behavior, the laser shock peening process may alter the profile of the stress-strain curve of these structures (e.g., when they are made from super-elastic materials such as super-elastic nickel-titanium alloy) so that the profile approaches linear-elastic behavior. This may improve the pushability, torquability, fatigue life, and the like of these structures or of the devices bearing these structures. The linear-elastic characteristics may be limited to a selected portion of the structure (i.e., laser shock peening to a selected portion of a structure may impart linear-elastic properties to that selected portion) or to essentially the entire structure. In some embodiments, further variations may be achieved by modulating the intensity of the shock wave (e.g., by modulation of laser intensity and/or through the selection of the sacrificial overlay materials, properties, and/or thickness) that the structures are subjected to, thereby modulating the depth of penetration of the cold work into the structure, for example. The amount of modulation can be manipulated to increase or decrease the degree to which elastic properties are affected.

Laser shock peening may also be used to increase the recoverable (elastic) strain in structures made from materials other than nickel-titanium alloys by cold working these components. For example, laser shock peening may increase the recoverable strain in structures made from or otherwise including materials such as stainless steel, platinum, other metals and/or metal alloys, and the like including any of those materials disclosed herein. This may improve the durability (e.g., resistance to kinking) of these structures.

Laser shock peening may also be useful in achieving desirable elastic strain behavior in delicate metal structures that cannot be machined or otherwise fabricated with the desired properties already imparted in the material. This could occur in cases where processing steps cause full or partial annealing of the material, where the material is more difficult or impossible to process when it possesses its desired final properties, or where the material is not commercially available with the desired final properties. For example, when a metal structure is laser cut, the laser cutting process may cause full or partial annealing of the structure. In this example, laser shock peening may be used to restore improved elastic properties to the structure after the laser cutting is complete.

As will be appreciated by those of skill in the art and others, the particular structure and assembly of the medical devices disclosed herein are provided by way of example only, and that many of a broad variety of others may be used. Having thus described several example embodiments of the present invention, those of skill in the art will readily appreciate that other embodiments may be made and used which fall within the scope of the claims attached hereto. Numerous advantages of the invention covered by this document have been set forth in the foregoing description. It will be understood that this disclosure is, in many respects, only illustrative. Changes may be made in details, particularly in matters of shape, size and arrangement of parts without exceeding the scope of the invention.

What is claimed is:

1. A medical device configured to navigate through anatomy, the device comprising:
an elongate shaft having a proximal end and a distal end;
a plurality of slots cut into the shaft to improve bending flexibility; and
wherein the elongate shaft includes at least one compressive residual stress region.

2. The medical device of claim 1, wherein the elongate shaft further comprises a plurality of beam sections.

3. The medical device of claim 2, wherein the beam sections are located between adjacent slots.

4. The medical device of claim 3, wherein the beam sections comprise an integral portion of the elongate shaft.

5. The medical device of claim 3, wherein the beam sections are a portion of the elongate shaft remaining after the plurality of slots are cut into the shaft.

6. The medical device of claim 3, wherein the at least one compressive residual stress region is located within one or more of the plurality of beam sections.

7. The medical device of claim 1, wherein the at least one compressive residual stress region is formed by a laser shock peening process.

8. The medical device of claim 1, wherein the at least one compressive residual stress region extends substantially from the proximal end to the distal end of the elongate shaft.

9. A medical device configured to navigate through anatomy, the device comprising:
an elongate shaft having a proximal end and a distal end;
a plurality of slots disposed along at least a portion of the elongate shaft, the slots providing increased flexibility in bending;
a plurality of segments positioned between the plurality of slots, the segments providing integrity to the elongate shaft; and
a first compressive residual stress region located within one or more of the plurality of segments.

10. The medical device of claim 9, wherein the plurality of segments include the first compressive residual stress region.

11. The medical device of claim 9, further comprising a second compressive residual stress region located along at least a portion of the elongate shaft.

12. The medical device of claim 11, wherein the second compressive residual stress region is located along the portion of the elongate shaft having the plurality of slots.

13. The medical device of claim 12, wherein the first compressive residual stress region has a first residual stress having a first magnitude and the second compressive residual stress region has a second residual stress having a second magnitude less than or equal to the first magnitude.

14. The medical device of claim 12, wherein the first compressive residual stress region has a first residual stress having a first magnitude and the second compressive residual stress region has a second residual stress having a second magnitude less than the first magnitude.

15. The medical device of claim 14, wherein the plurality of segments include the first compressive residual stress region.

16. A method of forming a medical device, the method comprising:
providing an elongate shaft having a proximal end and a distal end;
cutting a plurality of slots in at least a portion of the elongate shaft; and
forming compressive residual stresses in at least a portion of the elongate shaft.

17. The method of claim 16, further comprising subjecting at least a portion of the elongate shaft to a shock wave, wherein the shock wave forms compressive residual stresses in at least a portion of the elongate shaft.

18. The method of claim 16, wherein the forming of compressive residual stresses includes laser shock peening at least a portion of the elongate shaft.

19. The method of claim 16, wherein the compressive residual stresses oppose an applied tensile stress.

20. The method of claim 16, wherein the compressive residual stresses are formed below a surface of the elongate shaft.

21. The method of claim 20, wherein the compressive residual stresses are formed up to 1.5 mm below the surface of the elongate shaft.

22. The method of claim 16, wherein the step of cutting a plurality of slots defines a plurality of segments remaining between adjacent slots.

23. The method of claim 22, wherein the plurality of segments retain the integrity of the elongate shaft.

24. The method of claim 22, wherein the compressive residual stresses are formed in the plurality of segments.

25. The method of claim 24, wherein the compressive residual stresses formed in the plurality of segments provide the elongate shaft with increased elasticity and fatigue strength.

26. The method of claim 18, wherein the forming of compressive residual stresses through laser shock peening alters the elastic behavior of the portion of the elongated shaft.

27. The method of claim 26, wherein the elastic behavior of the portion of the elongated shaft is altered from a super-elastic elastic behavior to a profile that is more a linear-elastic behavior.

28. An elongated tubular member for use in a medical device, the tubular member comprising:
a metallic tubular body portion including a wall having a plurality of slots formed therein, the slots defining a connected ring structure within the body including a plurality of rings interconnected by one or more axial beams, wherein the body includes one or more compressive residual stress regions.

29. A medical device comprising:
an elongated metallic tubular member including a plurality of slots formed therein, at least a portion of the metallic tubular member being laser shock peened such that it includes one or more compressive residual stress regions.

30. The medical device of claim 29, wherein the device comprises a guidewire.

31. The medical device of claim 29, wherein the device comprises a catheter.

32. A guidewire, comprising:
an elongate core wire having a proximal section and a distal section; and
wherein the distal section includes one or more compressive residual stress regions.

33. The guidewire of claim 32, wherein the one or more compressive residual stress regions are formed by a laser shock peening process.

34. The guidewire of claim 32, wherein the distal section includes a distal region having a reduced diameter relative to the remainder of the core wire, wherein the distal region includes one or more compressive residual stress regions.

35. A method for manufacturing a medical device, the method comprising:
providing an elongate tubular member, the tubular member including a nickel-titanium alloy;
laser shocking peening at least a portion of the tubular member to alter the elastic properties of the tubular member;
incorporating the tubular member into the medical device.

36. The method of claim 35, wherein the tubular member initially comprises a super-elastic nickel-titanium alloy, and laser shocking peening at least a portion of the tubular member to alter the elastic properties of the tubular member includes converting the portion of the tubular member from a super-elastic alloy to an alloy having more linear elastic characteristics than the initial super-elastic nickel-titanium alloy.

37. A medical device, comprising:
an elongate tubular member having a plurality of slots formed therein, the tubular member having a first region having elastic properties and a second portion that is laser shock peened so that the second portion has different elastic properties than the first region.

38. The medical device of claim 37, wherein the first region has super-elastic properties and the second portion is laser shock peened so that the second portion has more linear-elastic properties than the first region.
